(19) United States
(12) Patent Application Publication
(10) Pub. No.: US 2008/0051649 A1
(43) Pub. Date: Feb. 28, 2008

(54) Prediction and treatment of brain tumor spread using MRI and external beam radiation

(76) Inventors: Walter G. O'Dell, Ontario, NY (US); Paul Okunieff, Rochester, NY (US); Anitha Priya Krishnan, Rochester, NY (US); Isaac Asher, Rochester, NY (US)

Correspondence Address: BLANK ROME LLP, 600 NEW HAMPSHIRE AVENUE, N.W., WASHINGTON, DC, 20037, US

(21) Appl. No.: 11/878,639

(22) Filed: Jul. 25, 2007

Related U.S. Application Data
(60) Provisional application No. 60/832,958, filed on Jul. 25, 2006.

(51) Int. Cl. A61B 5/055

(52) U.S. Cl. 600/410

(57) ABSTRACT

The invention is based on the realization that brain cancer cells spread preferentially along paths of elevated water diffusion, such as along nerve fiber bundles, that can be measured by magnetic resonance (MR) diffusion-weighted imaging (DWI) and the migration of cancer cells away from the primary tumor can be predicted using computational models that incorporate DWI information. The invention therefore applies DWI and models cell migration to develop appropriate non-symmetric margins for radiation treatment of malignant brain tumors.

### REFERENCE TO RELATED APPLICATION

The present application claims the benefit of U.S. Provisional Patent Application No. 60/832,958, filed Jul. 25, 2006, whose disclosure is hereby incorporated by reference in its entirety into the present disclosure.

### FIELD OF THE INVENTION

The invention is directed to a system and method for predicting tumor spread and migration in the brain and thereby improving clinical outcomes by changing the planning approach to radiotherapy and radiosurgery of brain cancer.

### DESCRIPTION OF RELATED ART

Several common types of primary and secondary brain cancer have a historical and physiological basis for aggressive tumor spread in the brain that thwarts curative treatment using our most sophisticated technology and all existing pharmacologic agents. Aggressive primary brain cancers are usually associated with oligodendrogliomas, low-grade astrocytomas, anaplastic astrocytomas, and glioblastomas. At present, the 5-year survival rate for patients of age 45+ ranges from 16% for those with anaplastic astrocytomas to 2% or less for those with glioblastomas. A recent RTOG study found that stereotactic radiotherapy (SRT) currently achieves a low 9% local control rate for glioblastomas.

Stereotactic radiotherapy (SRT) is used to deliver a large, lethal dose of radiation to a brain lesion with rapid dose falloff into the surrounding normal tissue. SRT is the treatment method of choice for lesions that cannot be readily accessed with conventional surgery. Typically, an SRT treatment plan of high-grade astrocytoma includes a margin of up to 2 cm surrounding the lesion to account for any unobserved, microscopic spread of the primary tumor. This margin size is selected based on histological analysis of tumor spread dating from the 1980's and in consideration of the critical need to minimize margin size to avoid potentially life-threatening complications resulting from radiation damage to surrounding healthy brain tissue. If the margin is inadequate then distant recurrences will occur.

Despite the symmetric 2 cm margin to account for unobserved, microscopic dispersal of cancer cells, recurrent tumors often occur. Current methods for predicting patterns of cancer spread are simply inadequate. A 2 cm margin is clearly too large in some directions leading to complication and loss of cognitive function. It is too small in others leading to recurrences, usually with a catastrophic result.

Diffusion weighting is a magnetic resonance imaging technique in which the image contrast is altered based on the diffusivity of water molecules within each pixel of the image. In any one experiment one can quantify the local diffusion coefficient along a predefined direction, where the direction is governed by the applied magnetic field gradients—the diffusion encoding gradients. By applying the diffusion encoding gradients along multiple directions, one unique direction for each scan, a diffusion coefficient unique for each direction is measured. By combining the information from multiple diffusion scans, one can reconstruct for each pixel in the image the three-dimensional (3D) diffusion coefficient tensor (a symmetric 3×3 matrix that is unique for each image pixel). This procedure is called diffusion tensor imaging—DTI. The tensor is diagonalized to obtain the three diffusion coefficient Eigenvalues and Eigen vectors. The direction of maximal diffusion is given by the Eigen vector corresponding to the maximal Eigen diffusion coefficient and is associated with the orientation of the most prominent fiber bundle. No injected contrast media nor any other invasive procedure nor any particularly special MR hardware is needed to obtain the DWI (diffusion weighted imaging) data, as it requires only a special sequence of commands to run the MR scanner to obtain the correct diffusion encoding steps. Post-acquisition analysis of the diffusion image data can be performed off-line to compute the unique diffusion tensor for each pixel in the series of brain slices.

The classic diffusion tensor approach has a significant limitation in that it accounts for only a single fiber orientation within any volumetric image element (voxel). The model fails therefore in voxels that have fiber crossing, branching or severe bending. High Angular Resolution Diffusion Imaging (HARDI) methods have been developed in recent years to overcome this limitation. HARDI involves sampling the diffusion function along a high number of directions (usually >60) and with high b values (achieved with strong applied magnetic field gradients and long inter-pulse delay times to accentuate the alterations in the MR signal due to water diffusion). The underlying multi-fiber diffusion environment can then be reconstructed as either a superposition of multiple non-coplanar diffusion tensors or using model-free approaches.

As early as 1961, post-mortem histological analyses in humans have suggested that glioma cells migrate preferentially along white matter tracts. More recently, human glioma cells implanted in the rat brain have been observed to move actively along the myelinated fibers of corpus callosum. En masse invasion occurs through both gray and white matter while migration of individual cells occurs preferentially through nerve fiber bundles. During embryogenesis neonatal astrocytes show a preferential movement along developing axon tracts. Thus there is existing evidence that migration of both healthy and cancerous astrocytes is influenced by the underlying fiber architecture.

The possible role of diffusive cell migration in human brain tissue has been simulated by previous researchers through retrospective analysis of diseased brains with massive tumor growth. The role of diffusion anisotropy in cell migration in the brain has been simulated by previous researchers by superposing a DWI dataset from a healthy human subject to brains of diseased subjects to estimate nonuniform growth patterns and compared the results to growth of real tumors. Other previous research has investigated the utility of DWI for: 1) assessing an index of relative diffusion anisotropy to discern white matter disruption due to the presence tumor infiltration, 2) differentiating tumor recurrence and radiation injury after radiotherapy, and 3) predicting cell density and proliferation activity of glioblastomas. These prior studies are distinct from the current proposal in that the infiltration models considered merely expansive growth of the primary tumor rather than isolated cell migration to distant sitesand the technology at the time did not afford the investigators the ability to acquire MR DWI and anatomical data in the same patient subjects.

### SUMMARY OF THE INVENTION

In treating aggressive brain tumors with radiation we find that treatment often fails because cancer cells have migrated undetected great distances beyond the treatment area. There is therefore a need in the art for an improved prediction and treatment for brain cancer spread. It is therefore an object of the invention to provide such improvements.

The invention is based on the realization that brain cancer cells spread preferentially along paths of elevated water diffusion, such as along nerve fiber bundles, that can be measured by magnetic resonance (MR) diffusion-weighted imaging (DWI) and the migration of cancer cells away from the primary tumor can be predicted using computational models that incorporate DWI information. The invention therefore applies DWI to develop appropriate non-symmetric margins for radiation treatment of malignant brain tumors. The invention can additionally apply a computational model of cell migration to better predict directions of microscopic tumor dispersal at the time of the initial treatment of the primary tumor and thereby enable us to tailor treatment margins to encompass the high-risk regions (thereby improving cancer control) while diminishing the margin in low-risk regions (thereby reducing harmful side-effects). The invention provides the first prospective analysis of tumor recurrence and DWI in brain cancer patients, and also involves the first combined analysis of tumor dispersal, DWI and histology in an animal model. Achievement of these aims marks a significant contribution to the treatment of brain cancer using SRS and allow for an innovative integration of novel MRI methodologies with state-of-the-art radiation delivery technology for cancer treatment.

Evidence in the literature links tumor dispersion in the brain to the underlying nerve fiber bundles, and recent advances in MR diffusion-weighting imaging enables us to discern this fiber architecture non-invasively in both the clinical and research settings. We have observed clinically a key link between patterns of tumor recurrence following high-dose stereotactic radiation therapy (SRS) and analysis of MR DWI.

In one aspect of the invention, a computational model of cell migration is used in which the model is constrained by the MR DWI (diffusion tensor imaging) information. Thus, this is an extension, and specific example for implementation, of the use of MR DWI data for treatment planning.

### BRIEF DESCRIPTION OF THE DRAWINGS

A preferred embodiment of the invention will be set forth in detail with reference to the drawings, in which:

FIGS. 1A-1D show experimental results from one patient;

FIGS. 2A-2D show experimental results from another patient; and

FIG. 3 is a block diagram of a system on which the present invention can be implemented.

### DETAILED DESCRIPTION OF THE PREFERRED EMBODIMENT

A preferred embodiment of the invention will be set forth in detail with reference to the drawings, in which like reference numerals refer to like elements throughout.

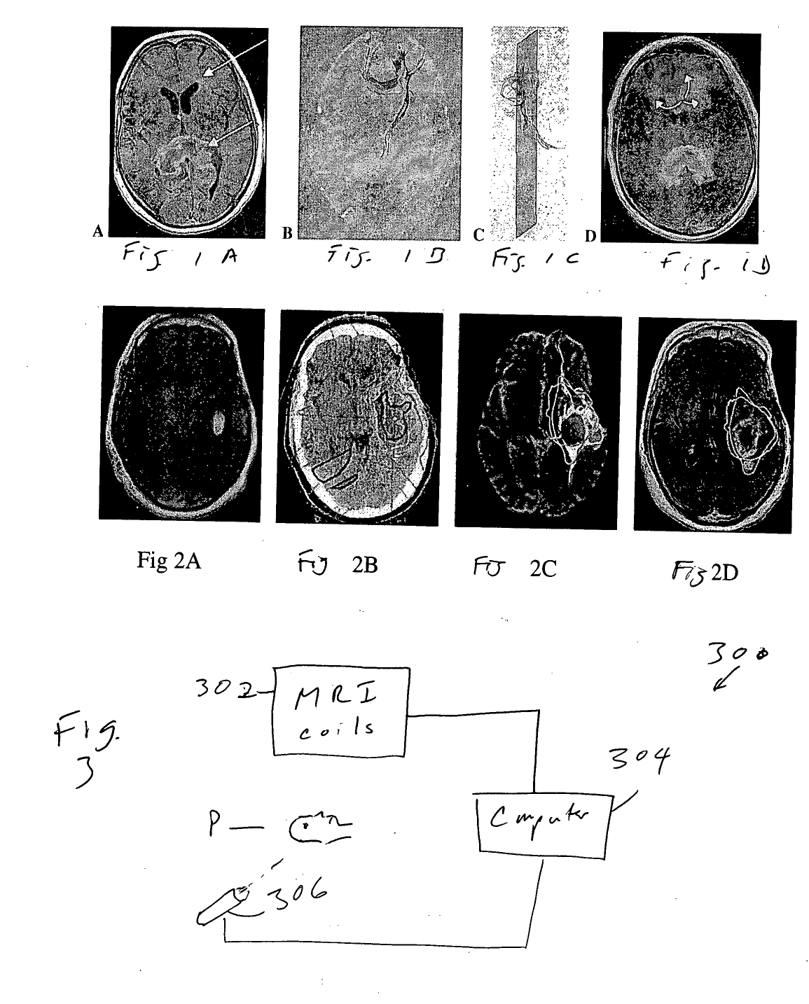

FIGS. 1A-D and 2A-D demonstrate our key preliminary results merging DWI tractography with repeated clinical follow-up of tumor spread and recurrence in high-risk subjects.

FIGS. 1A-1D show the following: FIG. 1A: Primary glioblastoma multiforme (GBM) in splenum of corpus callosum (green arrow) 6 months post-SRS treatment. Also seen at this time point is a small hyper-intense region in the anterior horn of the left lateral ventricle (white arrow), which proved to be a secondary tumor. FIG. 1B: T2 weighted image at the same time point with a depiction of all fibers emanating from the secondary tumor site. We employed a simple streamline approach (DTIstudio15]) to compute all fiber tracks passing through the secondary tumor site, showing several prominent fiber tracks coursing laterally and anteriorly from the secondary tumor site. FIG. 1C: An on-edge view of the slice plane gives a better appreciation of the 3D extent of the fiber tracks. In image B all the 3D fiber tracks are projected onto the plane of the slice. In C we see that the tracks directed posteriorly have also a significant out-of-plane component. FIG. 1D: Same subject 3 months later showing the spread of the secondary tumor, with substantial growth both laterally and anteriorly (yellow arrows). Thus the pattern of tumor expansion followed the dominate fiber tracts measured previously.

FIGS. 2A-2D show the following. FIG. 2A: and MR T1 weighted brain image of a patient with a glioblastoma in the right hemisphere. FIG. 2B: A CT image of the patient's brain depicting the radiation treatment plan used to treat this patient, where the contour lines represent different radiation dose exposure, with the highest doses toward the center of the tumor. FIG. 2C: The same MR T1 weighted image as in FIG. 2A but overlaid with 3 items. The wide contour represents the boundary of the lethal radiation dose exposure, taken from the radiation exposure data shown in FIG. 2B. Tissue within the wide contour line experience a lethal radiation dose. The white to red color rendering (shown in grayscale) represents the results of the computation model of cell migration, wherein the white (lightest) areas present the predicted highest concentration of cells after migration from the primary tumor. The yellow to red (darker) areas indicate predicted lesser concentration of cells. The narrow contour represents the results of a modified radiation treatment plan designed to encompass within the lethal radiation dose the areas of high predicted cell concentration that are also located within 15 mm of the originally planned lethal zone (wide contour). FIG. 2D: A follow-up MR image showing the presence of a recurrent tumor (just below the original tumor). The contours are the same as those of FIG. 2C. Of note, the recurrent tumor is located just outside the originally planned lethal zone (pink) but within the lethal zone that would have been used were the MR DWI data incorporated into the treatment planning process. Previous groups have modeled the local metastatic and glioma spread as a random mechanical walk with larger step size along paths of elevated water diffusion relative to the step sizes in the other directions. One realization of the present invention uses a constrained random walk of cells as a probabilistic model of local metastatic and glioma spread and supports the use of DWI and computational modeling as a means to predict and thereby ablate microscopic islands of migrating cells at the edge of the conventional planning target volume.

In the example of one realization of the present invention, the ratio of the rates of migration of cancer cells along white matter tracts versus gray matter is more dramatic than that observed for the diffusion of water molecules. Our objective in this realization of the invention is to model the relationship between the diffusivity of water molecules and migratory behavior of cancer cells in the brain. We use the single tensor transformation given by:
D̅=a₁(r)λ₁e₁e₁^{T}+a₂(r)λ₂e₂e₂^{T}+a₃(r)λ₃e₃e₃^{T} (1)

where aᵢ is defined by

$$[ \begin{matrix}{a}_{1} \\ {a}_{2} \\ {a}_{3}\end{matrix} ] = [ \begin{matrix}r & r & 1 \\ 1 & r & 1 \\ 1 & 1 & 1\end{matrix} ] [ \begin{matrix}{c}_{1} \\ {c}_{p} \\ {c}_{s}\end{matrix} ] \\ {c}_{1} = \frac{{λ}_{1} - {λ}_{2}}{{λ}_{1} + {λ}_{2} + {λ}_{3}} ; {c}_{s} = \frac{2 ( {λ}_{2} - {λ}_{3} )}{{λ}_{1} + {λ}_{2} + {λ}_{3}} ; {c}_{p} = \frac{3 {λ}_{3}}{{λ}_{1} + {λ}_{2} + {λ}_{3}} \qquad ( 2 )$$

The relationship between water diffusion and cell migration is controlled by the factor r. In voxels that have two crossing fibers the principal directions will be weighted by the volume fraction of each fiber bundle. The resulting cell migration probability map is compared to the measured cell migration indices obtained from the mouse histological studies, and the r and aᵢ parameters is optimized accordingly for the mouse model.

Our initial realization of the computational model of cancer cell migration is a modified random walk, starting with multiple seed locations within the tumor of interest in the human subjects. The model takes into account the two major biological phenomena underlying the spread of glioma and cells: growth and migration. Migration is considered to be anisotropic with cells migrating preferentially along a direction favored by direction of maximal diffusivity—along the white matter fibers. Prior studies have shown that the logistic model may be inadequate to model tumor growth; therefore, we use Gompertz law to model tumor growth. Tumor growth due to cell division will be represented by a differential equation in time.

$$\partial c / \partial t = pc ln ( \frac{{C}_{m}}{C} ) \qquad ( 3 )$$

where c is the tumor cell concentration, ƒ is a function representing the temporal evolution pattern of growth, ρ is the relative increase of cell concentration per unit time and cₘ is the initial cell concentration (10⁵ cells/mm³). The second part of the model takes into account the migration of tumor cells in space. The overall partial differential equation combines cell proliferation (time component) and cell infiltration (space component).

$$\partial c / \partial t = ∇ \cdot ( D ( x ) ∇ c ) + pc ln ( \frac{{C}_{m}}{C} ) \qquad ( 4 )$$

where ∇ is the gradient operator and D is the 3×3 diffusion tensor. The initial condition will be defined as c(0,x)=c₀(x). Boundary conditions are imposed based on the anatomic MR images to inhibit migration of cells through the dura covering the brain: D(x)∇c·n=0 for x on the sulcal and ventricular boundary of the brain, where n is the normal to the surface. Initial conditions will be represented by tumor cell concentration c₀ in each pixel, as selected manually on the anatomical images that represent in humans the primary site of GBM or metastases; and in mice the site of xenotransplantation. The computational model is constructed in Matlab.

The above model is customized to model tumor growth and cell migration via a Monte-Carlo approach incorporating fiber probability. Briefly, rather than considering the diffusivity within a pixel with a single Eigenvector, the surrounding diffusion environment is incorporated into a probability model of the distribution of fiber tracts contained within each pixel. Assuming that the distribution of fiber tract directions within each pixel can be considered as single or bi-Gaussian, then a combined Monte-Carlo and random-walk simulation can be used to estimate the probability of a given cell migrating to a predetermined location distal to the starting pixel location. The Monte-Carlo feature is to simulate 1000-5000 unique trajectories, using for each run a random number generator confined to obey the DWI-determined bi-Gaussian probability distribution for fiber direction. The simulated cell then steps a small increment along that direction, and then the local fiber trajectory is recomputed—the random-walk component. Standard statistical analyses using subgroups are used to assess the appropriateness of the step size and of the number of Monte-Carlo runs needed to achieve a meaningful result.

The distance metrics are used to identify the appropriate correspondence between the coefficients of water diffusion and the migration rates of cancer cells (the r parameter in Equation 2). The Monte-Carlo simulation is run using this parameter to generate between 1000-5000 model cell migratory pathways. A stopping time for the runs is matched to the 21-day interval between the injection of the U87 cells and the time of brain fixation. For a representative collection of U87 cells in the mouse brain, identified by histology and categorized by final location, each cell is matched to the nearest simulated cell trajectory. The migratory distances (preserving sign) between the two sets of matched cells are compiled and recorded for each real cell and the data analyzed using standard statistical means to determine the presence of a consistent bias (overshoot or undershoot) of the simulation (by consideration of the mean miss distance), and the accuracy of the model (by consideration of the standard deviations around the mean miss distance). If the bias is nonnegligible, then the r parameter in Equation 2 can be adjusted and the simulation repeated until a zero, or nearly zero, bias is obtained. A value for the standard deviation that is less than 25% of the mean distance traveled for each cell is used to indicate the success or failure of the computational model. Failure of the computational model necessitates the incorporation of additional complexity to the fiber reconstruction approach and to the cell infiltration model (Equation 4).

FIG. 3 shows a block diagram of a system 300 on which the preferred embodiment can be implemented. MRI coils 302 image a region of interest in the brain of a patient P. A computer 304, which can be any suitable computing device, receives raw data signals from the coils and performs the calculations described above to control a radiosurgery device 306.

While a preferred embodiment of the present invention has been set forth above, those skilled in the art who have reviewed the present disclosure will readily appreciate that other embodiments can be realized within the scope of the invention. For example, numerical values are illustrative rather than limiting, as are specific computational techniques. Therefore, the present invention should be construed as limited only by the appended claims.

We claim:

1. A method for predicting a spread of brain cancer, the method comprising:
(a) taking magnetic resonance,imaging data of the brain anatomy;
(b) taking diffusion-weighted magnetic resonance image data of the brain; and
(c) from the diffusion-weighted image data and a model of cancer cell migration, predicting the spread of the brain cancer.

2. The method of claim 1, wherein step (a) is performed using high angular resolution diffusion imaging (HARDI).

3. The method of claim 1, wherein step (c) comprises predicting the spread of the brain cancer along nerve fiber tracts.

4. The method of claim 3, wherein the spread of the brain cancer along the nerve fiber tracts is predicted by using a persistent angular structure method.

5. The method of claim 4, wherein the spread of the brain cancer is predicted by using coefficients of water diffusion.

6. The method of claim 5, wherein a probability that a given cell will migrate to a given location is determined using a computational model

7. The method of claim 6, wherein the computational model comprises a constrained random walk.

8. A method for predicting a spread of brain cancer and treating the brain cancer, the method comprising:
(a) taking magnetic resonance imaging data of the brain anatomy;
(b) taking diffusion-weighted magnetic resonance image data;
(c) from the diffusion-weighted image data and a model of cancer cell migration, predicting the spread of the brain cancer; and
(d) applying a treatment to the brain cancer in accordance with the spread predicted in step (c).

9. The method of claim 8, wherein step (a) is performed using high angular resolution diffusion imaging.

10. The method of claim 8, wherein step (c) comprises predicting the spread of the brain cancer along nerve fiber tracts.

11. The method of claim 10, wherein the spread of the brain cancer along the nerve fiber tracts is predicted by using a persistent angular structure method.

12. The method of claim 11, wherein the spread of the brain cancer is predicted by using coefficients of water diffusion.

13. The method of claim 12, wherein a probability that a given cell will migrate to a given location is determined using a computational model.

14. The method of claim 13, wherein the computational model comprises a constrained random walk.

15. The method of claim 8, wherein step (d) is performed using a radiation beam.

16. A system for predicting a spread of brain cancer, the system comprising:
a magnetic resonance imaging device for taking magnetic resonance imaging data of the brain anatomy and of the brain diffusion environment; and
a processing device, in communication with the magnetic resonance imaging device, for forming a diffusion-weighted image from the magnetic resonance imaging data and from the diffusion-weighted image and a model of cancer cell migration, predicting the spread of the brain cancer.

17. The system of claim 16, wherein the magnetic resonance imaging device uses high angular resolution diffusion imaging.

18. The system of claim 16, wherein the processing device predicts the spread of the brain cancer along nerve fiber tracts.

19. The system of claim 18, wherein the spread of the brain cancer along the nerve fiber tracts is predicted by using a persistent angular structure method.

20. The system of claim 19, wherein the spread of the brain cancer is predicted by using coefficients of water diffusion.

21. The system of claim 20, wherein a probability that a given cell will migrate to a given location is determined using a computational model.

22. The system of claim 21, wherein the computational model comprises a constrained random walk.

23. A system for predicting a spread of brain cancer and treating the brain cancer, the system comprising:
a magnetic resonance imaging device for taking magnetic resonance imaging data of the brain cancer; and
a processing device, in communication with the magnetic resonance imaging device, for forming a diffusion-weighted image from the magnetic resonance imaging data, and from the diffusion-weighted image and a model of cancer cell migration, predicting the spread of the brain cancer; and
a treatment device for applying a treatment to the brain cancer in accordance with the predicted spread.

24. The system of claim 23, wherein the magnetic resonance imaging device uses high angular resolution diffusion imaging.

25. The system of claim 24, wherein the processing device predicts the spread of the brain cancer along nerve fiber tracts.

26. The system of claim 25, wherein the spread of the brain cancer along the nerve fiber tracts is predicted by using a persistent angular structure method.

27. The system of claim 26, wherein the spread of the brain cancer is predicted by using coefficients of water diffusion.

28. The system of claim 27, wherein a probability that a given cell will migrate to a given location is determined using a computational model.

29. The system of claim 28, wherein the computational model comprises a constrained random walk.

30. The system of claim 23, wherein the treatment device emits a beam of radiation at the brain cancer.
